(19) United States
(12) Patent Application Publication
(10) Pub. No.: US 2024/0065903 A1
(43) Pub. Date: Feb. 29, 2024

(54) Sterile Bandage Dispenser

(71) Applicant: Christopher Dale Coon, Missouri City, TX (US)

(72) Inventor: Christopher Dale Coon, Missouri City, TX (US)

(21) Appl. No.: 18/238,758

(22) Filed: Aug. 28, 2023

Related U.S. Application Data
(60) Provisional application No. 63/401,380, filed on Aug. 26, 2022.

(51) Int. Cl. A61F 15/00; B65H 37/00

(52) U.S. Cl. CPC A61F 15/002; B65H 37/002

(57) ABSTRACT

Apparatuses and methods for storing and dispensing a continuous feed of adhesive bandages. In one embodiment, the apparatus comprises a housing, a cover, and a plurality of rotating elements which may comprise one or more spools and one or more rollers, wherein a continuous feed of bagged adhesive bandages may be dispensed from a spooled loading system. In one embodiment, the apparatus comprises a housing, a cover, a plurality of rotating elements which may comprise one or more spools and one or more rollers, and a bandage magazine, wherein a continuous feed of bagged adhesive bandages may be dispensed from a magazine loading system. Two embodiments of a method are presented which correspond to each embodiment of the apparatuses, and comprise individual steps of loading, storing, and dispensing a plurality of bagged adhesive bandages, wherein the dispensing further comprises individual steps of separating, removing, and collecting a bag within which an adhesive bandage may be stored.

### CROSS-REFERENCE TO RELATED APPLICATIONS

This application is a non-provisional application that claims the benefit of U.S. Provisional Application No. 63/401,380 filed on Aug. 26, 2022, the disclosure of which is incorporated by reference herein in its entirety.

### STATEMENT REGARDING FEDERALLY SPONSORED RESEARCH OR DEVELOPMENT

Not applicable.

### BACKGROUND OF THE INVENTION

### Field of the Invention

The present invention relates methods and apparatuses for storing and dispensing adhesive bandages. More particularly, the present invention relates to methods and apparatuses for storing and dispensing a continuous feed of adhesive bandages which enable single-handed application of an adhesive bandage.

### Background of the Invention

Adhesive bandages are a widely available and well known component of typical first aid care practices applied to safely and cleanly treat and protect common cuts and wounds. Persons wishing to be prepared for emergencies may store a supply of adhesive bandages in first aid kits or home medicine cabinets, and commercial, retail, or industrial facilities may desire or may be regulated to have a variety of emergency first aid products including adhesive bandages available for persons working in or visiting those facilities. Adhesive bandages are available in a wide variety of shapes including but not limited to strips, spots, patches, butterfly, and other shapes for specialty applications such as fingers or knuckles, each of which may be produced in a variety of sizes. Several manufacturers produce adhesive bandages, which are often sold in packages as a quantity of a specific shape and size, in variety packs which may include quantities of different shapes and sizes, or in combination with other emergency-related products as part of a first-aid kit.

Initially developed in the early twentieth century, a typical adhesive bandage may be formed from four basic components, including an adhesive strip or sheet, a non-adhesive pad which is typically absorbent, a removable backing which prevents the bandage from adhering to its packaging, and a bag or wrap surrounding the bandage which may also serve to keep the bandage sterile. In a common example, an adhesive bandage may be formed from a fabric strip having an adhesive material on one side, a cotton pad located about centrally on the adhesive side of the strip, a two-part, overlapping coated paper backing fully covering the cotton pad and adhesive side of the strip, and a coated paper bag surrounding the bandage which may be provided with one or more tabs which can be pulled in order to tear the bag open in order to expose the bandage. Adhesive bandages may also be formed from a range of materials, for example the adhesive strip or sheet may be formed from plastic, latex, foam, or tricot, the non-adhesive pad may be formed from other absorbent materials or may be configured to apply or release a topical or non-topical medicine, and the backing, bag, or wrap may be formed from a suitable coated or non-coated plastic material.

In a typical application, a person who may have sustained a wound may need to locate a package if individually wrapped adhesive bandages, remove a single wrapped bandage from the package, open the bag surrounding the bandage, remove each of the two overlapping backing strips, and then apply the adhesive bandage over the wound. In this typical application, the step of opening the bag may include pulling on two available tabs of the bag intended to assist in tearing the bag open, which commonly involves gripping each tab with a thumb and one or more fingers of each hand. Additionally, the step of removing the overlapping backing strips may involve holding the bandage with one hand while simultaneously manipulating the bandage into a semi-folded orientation with a second hand and locating, exposing, and pulling an exposed portion of the first of the two overlapping backing strips, and then changing the way in which the bandage is held in order to be able to locate, expose, and pull on a portion of the second of the two backing materials.

Indeed, such a typical application may necessitate the availability of two hands, which may be problematic for the person if the wound were sustained on an arm, wrist, hand, knuckle, or finger. Likewise, the process just described may be problematic where, for example, the person may have a need to apply pressure to the wound with one hand or otherwise not have two hands available to prepare the bandage for application to the wound. Consequently, there is a need for apparatuses and methods for storing and dispensing adhesive bandages which enable convenient, single-handed application of an adhesive bandage without interfering with the sterile nature of the adhesive bandage's packaging.

### BRIEF SUMMARY OF SOME OF THE PREFERRED EMBODIMENTS

These and other needs in the art are addressed in one embodiment by a continuous-feed bandage dispenser which may principally comprise a housing, a cover, and a plurality of rotating elements which may comprise one or more spools and one or more rollers, wherein a continuous feed of bagged adhesive bandages may be dispensed from a spooled loading system.

These and other needs in the art are addressed in one embodiment by a continuous-feed bandage dispenser which may principally comprise a housing, a cover, a plurality of rotating elements which may comprise one or more spools and one or more rollers, and a bandage magazine, wherein a continuous feed of bagged adhesive bandages may be dispensed from a magazine loading system.

These and other needs in the art are addressed in one embodiment by a method of dispensing an individual adhesive bandage from a continuous feed of adhesive bandages, wherein a bagged adhesive bandage may be dispensed having its bag separated, removed, and collected internally within a continuous-feed bandage dispenser comprising a spooled loading system, with the dispensed adhesive bandage presented for single-handed application. The method may comprise individual steps of loading, storing, and dispensing a plurality of bagged adhesive bandages, wherein the dispensing may further comprise individual steps of separating, removing, and collecting a bag within which an adhesive bandage may be stored.

These and other needs in the art are addressed in one embodiment by a method of dispensing an individual adhesive bandage from a continuous feed of adhesive bandages, wherein a bagged adhesive bandage may be dispensed having its bag separated, removed, and collected internally within a continuous-feed bandage dispenser comprising a magazine loading system, with the dispensed adhesive bandage presented for single-handed application. The method may comprise individual steps of loading, storing, and dispensing a plurality of bagged adhesive bandages, wherein the dispensing may further comprise individual steps of separating, removing, and collecting a bag within which an adhesive bandage may be stored.

The foregoing has outlined rather broadly the features and technical advantages of the present invention in order that the detailed description of the invention that follows may be better understood. Additional features and advantages of the invention will be described hereinafter that form the subject of the claims of the invention. It should be appreciated by those skilled in the art that the conception and the specific embodiments disclosed may be readily utilized as a basis for modifying or designing other embodiments for carrying out the same purposes of the present invention. It should also be realized by those skilled in the art that such equivalent embodiments do not depart from the spirit and scope of the invention as set forth in the appended claims.

### BRIEF DESCRIPTION OF THE DRAWINGS

For a detailed description of the preferred embodiments of the invention, reference will now be made to the accompanying drawings in which:

FIG. 1A illustrates a first embodiment of a continuous-feed bandage dispenser comprising a spooled loading system;

FIG. 1B illustrates a first isometric view of a first embodiment of a continuous-feed bandage dispenser comprising a spooled loading system;

FIG. 1C illustrates a second isometric view of a first embodiment of a continuous-feed bandage dispenser comprising a spooled loading system;

FIG. 2A illustrates a second embodiment of a continuous-feed bandage dispenser comprising a magazine loading system;

FIG. 2B illustrates a first isometric view of a second embodiment of a continuous-feed bandage dispenser comprising a magazine loading system;

FIG. 2C illustrates a second isometric view of a second embodiment of a continuous-feed bandage dispenser comprising a magazine loading system;

FIG. 3A illustrates a continuous-feed path of a first embodiment of a continuous-feed bandage dispenser comprising a spooled loading system; and

FIG. 3B illustrates a continuous-feed path of a second embodiment of a continuous-feed bandage dispenser comprising a magazine loading system.

### DETAILED DESCRIPTION OF THE PREFERRED EMBODIMENTS

In the description that follows, two embodiments of a continuous-feed bandage dispenser apparatus are presented, followed thereafter by a detailed description of the methods by which each may enable single-handed dispensing and application of an adhesive bandage.

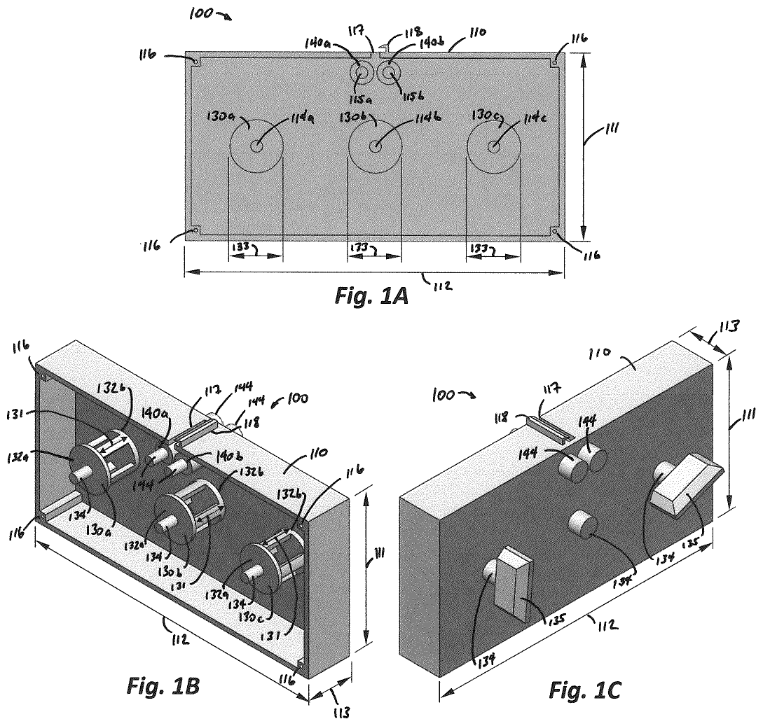

FIGS. 1A-1C illustrate an embodiment of bandage dispenser 100, which may principally comprise housing 110, cover 120 (not shown), and a plurality of rotating elements which may comprise one or more spools 130 and one or more rollers 140. As illustrated, bandage dispenser 100 is provided with three spools 130a,b,c and two rollers 140a,b.

Housing 110 may be formed having any shape and size which may be suitable for storing and providing a continuous feed of spooled adhesive bandages. For example, housing 110 may be formed having a shape of a cube, cuboid, cylinder, or other such suitable shape. In the embodiment shown, housing 110 is illustrated having a cuboid shape comprising an inner volume and a size having height 111, width 112, and depth 113. Depth 113 may be any depth suitable for accommodating spools 130 and rollers 140. Height 111 and width 112 may be any height and width suitable for accommodating outermost spools 130a,c when outermost spools 130a,c are fully loaded with remnant peeled bandage bag corresponding to an initial capacity of spooled adhesive bandages of innermost spool 130b. The inner volume of housing 110 may provide any capacity suitable for storing one or more loaded spools of spooled adhesive bandages and collecting one or more loaded spools of remnant peeled bandage bags. Housing 110 may be formed having side walls and a back plate, each having a thickness, and may be formed from separate components including individual side walls and a back plate, or may be formed as a single component, for example by hollowing-out a solid volume, or through any known additive manufacturing process, for example 3D printing. Housing 110 may be formed from any material suitable for sterile storage of medical grade products, for example polylactic acid (PLA), acrylonitrile butadiene styrene (ABS), polyvinyl alcohol plastic (PVA), polycarbonate (PC), stainless-steel, aluminum, carbon fiber, or combinations thereof.

Housing 110 may be provided with one or more apertures 114,115 for accommodating one or more rotating elements. In the embodiment shown, housing 110 may be provided with three apertures 114a,b,c for accommodating each of the one or more spools 130a,b,c, and two apertures 115a,b for accommodating each of the one or more rollers 140a,b, or a projection or axle each thereof. Housing 110 may be provided with additional apertures or bores for securing thereto cover 120 (not shown). In the embodiment of bandage dispenser 100 illustrated in FIGS. 1A-1C, housing 110 is provided with four bores 116 which may threadably receive fasteners (not shown) for securing cover 120 to housing 110. One or more side walls of housing 110 may be formed having dispensing port 117 for ejecting dispensed adhesive bandages in the method which will be described later. Additionally, the one or more side walls of housing 110 on which dispensing port 117 may be located may also feature separating projection 118 for separating a backing material from a dispensed adhesive bandage in the method which will be described later. In embodiments, apertures 115a,b may be located about dispensing port 117 so as to facilitate separation of an adhesive bandage bag and redirect a pathway of a continuous feed of remnant peeled bandage bags within housing 110, and the location of apertures 115a,b may correspond to one or more dimensions of an adhesive bandage.

Cover 120 (not shown) may be formed having any shape and size suitable for enclosing the volume of housing 110 to provide bandage dispenser 100 with an enclosed capacity. For example, cover 120 may be formed having a thickness and an overall shape which is square, rectangular, or circular, or other such suitable shape to be mated to housing 110. In the embodiment shown, cover 120 may be formed having an overall shape which is rectangular, and having a size which corresponds to height 111 and width 112. Cover 120 may be formed through any known manufacturing process, for example an additive manufacturing process such as 3D printing, and from any material suitable for sterile storage of medical grade products, for example polylactic acid (PLA), acrylonitrile butadiene styrene (ABS), polyvinyl alcohol plastic (PVA), polycarbonate (PC), stainless-steel, aluminum, carbon fiber, or combinations thereof, and may be fully transparent, partially transparent, partially opaque, or fully opaque. In embodiments, cover 120 may be transparent or may provide a window such that a person observing bandage dispenser 100 may ascertain or estimate a capacity of spooled adhesive bandages which may remain or may be available to be dispensed from bandage dispenser 100, or may allow the person to recognize any interferences, mechanical or otherwise, which may prevent bandage dispenser 100 from dispensing one or more adhesive bandages in the method which will be described later. Cover 120 may be provided with one or more apertures for accommodating one or more rotating elements. In the embodiment shown, cover 120 may be provided with three apertures for accommodating each of the three spools 130a,b,c, and two apertures for accommodating each of the two rollers 140a,b, or a projection or axle each thereof. Cover 120 may be provided with additional apertures for securing cover 120 to housing 110. In the embodiment of bandage dispenser 100 illustrated in FIGS. 1A-1C, cover 120 may be provided with four apertures through which fasteners (not shown) may secure cover 120 to housing 110.

The rotating elements of bandage dispenser 100 may comprise one or more spools 130 and one or more rollers 140, each of which may be generally cylindrical in shape, having a central axis, a length 131,141, and opposing planar surfaces 132a,b, 142a,b. In embodiments, spool 130 and roller 140 may be provided with a curved surface adjoining each of its opposing planar surfaces, and may be solid or hollow. In alternate embodiments, the curved surface of spool 130 and roller 140 may comprise one or more apertures, or may be formed as a frame of a curved surface, as illustrated in FIG. 1B. Length 131,141 may be any length suitable for rotatably disposing spool 130 and roller 140 between housing 110 and cover 120, and may correspond to one or more dimensions of an adhesive bandage. Each spool 130 may comprise a circumference defined by cross sectional diameter 133, and may be formed having, or may be provided with, a centrally located axle or projection 134 on at least one of the opposing planar surfaces 132a,b which may be adapted to be received into aperture 114 of housing 110 or a corresponding aperture of cover 120, and about which spool 130 may rotate. Similarly, each roller 140 may comprise a circumference defined by cross sectional diameter 143, and may be formed having, or may be provided with, a centrally located axle or projection 144 on at least one of the opposing planar surfaces 142a,b which may be adapted to be received into aperture 115 of housing 110 or a corresponding aperture of cover 120, and about which roller 140 may rotate. In alternate embodiments, in place of centrally located axel or projection 134,144, spool 130 or roller 140 may be provided with a centrally located bore suitable to receive a fastener through a corresponding aperture of housing 110 or cover 120, which may allow spool 130 or roller 140 to rotate about its central axis. Cross sectional diameter 133 of spool 130 may be any cross sectional diameter suitable for storing a capacity of spooled adhesive bandages within housing 110, or suitable for collecting a corresponding capacity of remnant peeled bandage bags within housing 110, and may correspond to one or more dimensions of an adhesive bandage. Cross sectional diameter 143 of roller 140 may be any cross sectional diameter suitable for redirecting a pathway of a continuous feed of remnant peeled bandage bags within housing 110, and may correspond to one or more dimensions of an adhesive bandage. Spool 130 and roller 140 may be formed through any known manufacturing process, for example an additive manufacturing process such as 3D printing, and from any material suitable for sterile storage of medical grade products, for example polylactic acid (PLA), acrylonitrile butadiene styrene (ABS), polyvinyl alcohol plastic (PVA), polycarbonate (PC), stainless-steel, aluminum, carbon fiber, or combinations thereof.

As illustrated in FIG. 1C, one or more spools 130 may be provided with handle 135 for manually causing spool 130 to rotate. In alternate embodiments, each of the one or more spools 130 may be caused to rotate by powered or automated form of rotation, or may be mechanically in communication with any of the one or more spools 130 such that a pulling force applied to an adhesive bandage at dispensing port 117 may cause at least one of the one or more spools 130 to rotate in synchronization with the rate at which the adhesive bandage is dispensed through dispensing port 117.

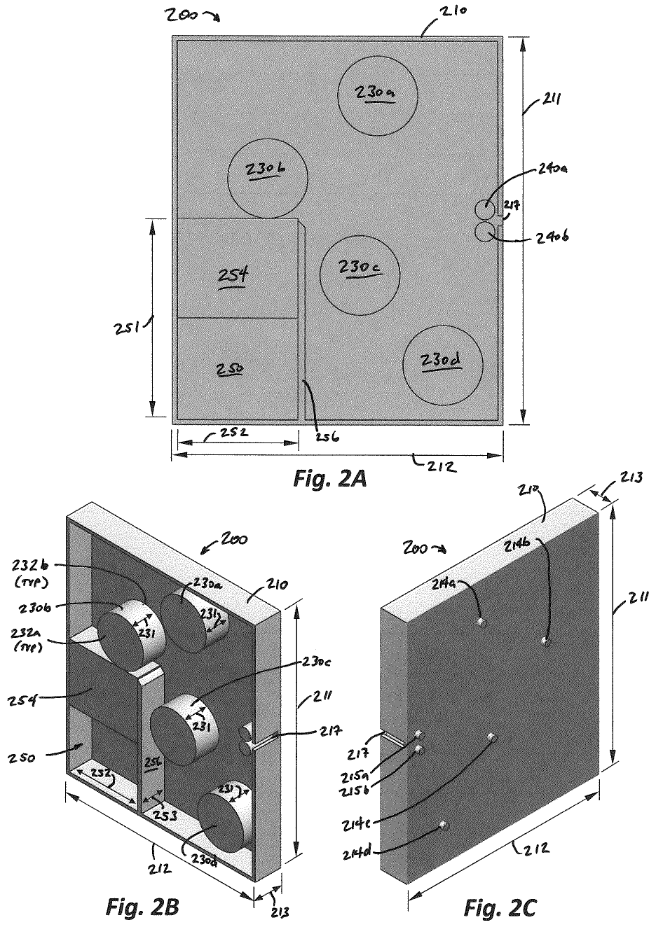

FIGS. 2A-2C illustrate an embodiment of bandage dispenser 200, which may principally comprise housing 210, cover 220 (not shown), a plurality of rotating elements which may comprise one or more spools 230, one or more rollers 240, and bandage magazine 250. As illustrated, bandage dispenser 200 is provided with four spools 230a,b,c,d and two rollers 240a,b.

Housing 210 may be formed having any shape and size which may be suitable for storing and providing a continuous feed of spooled adhesive bandages. For example, housing 210 may be formed having a shape of a cube, cuboid, cylinder, or other such suitable shape. In the embodiment shown, housing 210 is illustrated having a cuboid shape comprising an inner volume and a size having height 211, width 212, and depth 213. Depth 213 may be any depth suitable for accommodating spools 230, rollers 240, and bandage magazine 250. Height 211 and width 212 may be any height and width suitable for accommodating outermost spools 230a,d when outermost spools 230a,d are fully loaded with remnant peeled bandage bag corresponding to an initial capacity of adhesive bandages bandage magazine 250. The inner volume of housing 210 may provide any capacity suitable for storing one or more loaded magazines of adhesive bandages and collecting one or more loaded spools of remnant peeled bandage bags. Housing 210 may be formed having side walls and a back plate, each having a thickness, and may be formed from separate components including individual side walls and a back plate, or may be formed as a single component, for example by hollowing-out a solid volume, or through any known additive manufacturing process, for example 3D printing. Housing 210 may be formed from any material suitable for sterile storage of medical grade products, for example polylactic acid (PLA), acrylonitrile butadiene styrene (ABS), polyvinyl alcohol plastic (PVA), polycarbonate (PC), stainless-steel, aluminum, carbon fiber, or combinations thereof.

Housing 210 may be provided with one or more apertures 214,215 for accommodating one or more rotating elements. In the embodiment shown, housing 210 may be provided with four apertures 214a,b,c,d for accommodating each of the one or more spools 230a,b,c,d, and two apertures 215a,b for accommodating each of the one or more rollers 240a,b, or a projection or axle each thereof. Housing 210 may be provided with additional apertures or bores for securing thereto cover 220 (not shown). In the embodiment of bandage dispenser 200 illustrated in FIGS. 1A-1C, housing 210 may be provided with four bores 216 (not shown) which may threadably receive fasteners (not shown) for securing cover 220 to housing 210. One or more side walls of housing 210 may be formed having dispensing port 217 for ejecting dispensed adhesive bandages in the method which will be described later. Additionally, the one or more side walls of housing 210 on which dispensing port 217 may be located may also feature separating projection 218 (not shown) for separating a backing material from a dispensed adhesive bandage in the method which will be described later, which may be formed and may function similar to separating projection 118 of bandage dispenser 100. In embodiments, apertures 215a,b may be located about dispensing port 217 so as to facilitate separation of an adhesive bandage bag and redirect a pathway of a continuous feed of remnant peeled bandage bags within housing 210, and the location of apertures 215a,b may correspond to one or more dimensions of an adhesive bandage.

Cover 220 (not shown) may be formed having any shape and size suitable for enclosing the volume of housing 210 to provide bandage dispenser 200 with an enclosed capacity. For example, cover 220 may be formed having a thickness and an overall shape which is square, rectangular, or circular, or other such suitable shape to be mated to housing 210. In the embodiment shown, cover 220 may be formed having an overall shape which is rectangular, and having a size which corresponds to height 211 and width 212. Cover 220 may be formed through any known manufacturing process, for example an additive manufacturing process such as 3D printing, and from any material suitable for sterile storage of medical grade products, for example polylactic acid (PLA), acrylonitrile butadiene styrene (ABS), polyvinyl alcohol plastic (PVA), polycarbonate (PC), stainless-steel, aluminum, carbon fiber, or combinations thereof, and may be fully transparent, partially transparent, partially opaque, or fully opaque. In embodiments, cover 220 may be transparent or may provide a window such that a person observing bandage dispenser 200 may ascertain or estimate a capacity of spooled adhesive bandages which may remain or may be available to be dispensed from bandage dispenser 200, or may allow the person to recognize any interferences, mechanical or otherwise, which may prevent bandage dispenser 200 from dispensing one or more adhesive bandages in the method which will be described later. Cover 220 may be provided with one or more apertures for accommodating one or more rotating elements. For example, in embodiments, cover 220 may be provided with apertures for accommodating each of the spools 230, and apertures for accommodating each of two rollers 140, or a projection or axle each thereof. Cover 220 may be provided with additional apertures through which fasteners (not shown) may secure cover 220 to housing 210.

The rotating elements of bandage dispenser 200 may comprise one or more spools 230 and one or more rollers 240, each of which may be generally cylindrical in shape, having a central axis, a length 231, 241, and opposing planar surfaces 232a,b, 242a,b. In embodiments, spool 230 and roller 240 may be provided with a curved surface adjoining each of its opposing planar surfaces, and may be solid or hollow. In alternate embodiments, the curved surface of spool 230 and roller 240 may comprise one or more apertures, or may be formed as a frame of a curved surface. Length 231,241 may be any length suitable for rotatably disposing spool 230 and roller 240 between housing 210 and cover 220, and may correspond to one or more dimensions of an adhesive bandage, Each spool 230 may comprise a circumference defined by cross sectional diameter 233, and may be formed having, or may be provided with, a centrally located axle or projection 234 (not shown) on at least one of the opposing planar surfaces 232a,b which may be adapted to be received into aperture 214 of housing 210 or a corresponding aperture of cover 220, and about which spool 230 may rotate. Similarly, each roller 240 may comprise a circumference defined by cross sectional diameter 243, and may be formed having, or may be provided with, a centrally located axle or projection 244 (not shown) on at least one of the opposing planar surfaces 242a,b which may be adapted to be received into aperture 215 of housing 210 or a corresponding aperture of cover 220, and about which roller 240 may rotate. In alternate embodiments, in place of centrally located axel or projection 234,244, spool 230 or roller 240 may be provided with a centrally located bore suitable to receive a fastener through a corresponding aperture of housing 210 or cover 220, which may allow spool 230 or roller 240 to rotate about its central axis, Cross sectional diameter 233 of spool 230 may be any cross sectional diameter suitable for storing a capacity of spooled adhesive material within housing 210, or suitable for collecting a corresponding capacity of remnant peeled bandage bags within housing 210, and may correspond to one or more dimensions of an adhesive bandage. Cross sectional diameter 243 of roller 240 may be any cross sectional diameter suitable for redirecting a pathway of a continuous feed of remnant peeled bandage bags within housing 210, and may correspond to one or more dimensions of an adhesive bandage. Spool 230 and roller 240 may be formed through any known manufacturing process, for example an additive manufacturing process such as 3D printing, and from any material suitable for sterile storage of medical grade products, for example polylactic acid (PLA), acrylonitrile butadiene styrene (ABS), polyvinyl alcohol plastic (PVA), polycarbonate (PC), stainless-steel, aluminum, carbon fiber, or combinations thereof.

In embodiments, each of the one or more spools 230 may be caused to rotate manually, or by powered or automated form of rotation, or may be mechanically in communication with any of the one or more spools 230 such that a pulling force applied to an adhesive bandage at dispensing port 217 may at least one of the one or more spools 230 to rotate in synchronization with the rate at which the adhesive bandage is dispensed through dispensing port 217.

Bandage magazine 250 may be defined by a volume located within the volume formed by housing 220 and bandage magazine wall 256, and may be having internal height 251, internal width 252, and internal depth 253, each of which may correspond to one or more dimensions of an adhesive bandage. In embodiments, internal height 251, internal width 252, and internal depth 253 may be selected so as to volumetrically accommodate a stacked plurality of individual bagged adhesive bandages 254, each of which may be similarly oriented in space, such that an end of each bagged adhesive bandage which may be preferred to be opened first at dispensing port 217 may be located adjacent to a common internal surface of bandage magazine wall 256.

One or more biasing elements 255 (not shown) may be disposed within bandage magazine 250. In embodiments, biasing element 255 may be in contact with an internal surface of bandage magazine 250 and a surface of an individual bagged adhesive bandage located in a bottom-most position of stacked plurality of individual bagged adhesive bandages 254. In alternate embodiments, biasing element 255 may be in contact with an internal surface of bandage magazine 250 and a plate or shelf which may be in contact with a surface of an individual bagged adhesive bandage located in a bottom-most position of stacked plurality of individual bagged adhesive bandages 254. Biasing element 255 may function to bias stacked plurality of individual bagged adhesive bandages 254 toward and against the curved surface spool 230b such that spool 230b may contact an individual bagged adhesive bandage located in a top-most position of stacked plurality of individual bagged adhesive bandages 254. In this manner, as a top-most bagged adhesive bandage may be removed from the stacked plurality, biasing element 255 may cause the stacked plurality to rise in the internal magazine such that a next top-most may be biased toward and against the curved surface spool 230b. Biasing element 255 may thus be disposed within bandage magazine 250 in a compressed configuration, and provided with an effective biasing length which may allow a bottom-most bagged adhesive bandage of the stacked plurality to be biased against spool 230b after each of the preceding bagged adhesive bandages have been removed from the stacked plurality.

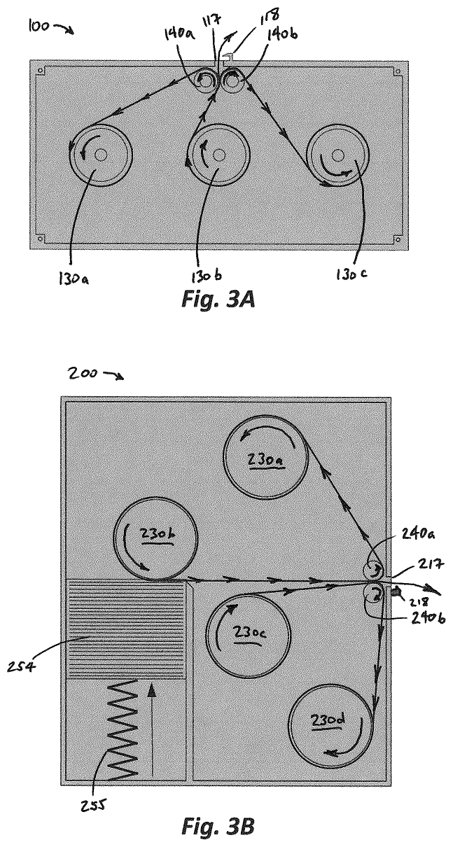

Referring now to FIGS. 3A and 3B, the methods through which a continuous feed of adhesive bandages may be dispensed from bandage dispenser 100,200 will now be described.

In the first embodiment illustrated by FIG. 3A, bandage dispenser 100 may be loaded with a plurality of adhesive bandages comprising spooled adhesive bandages. In embodiments, it may be necessary for the spooled adhesive bandages to first be prepared prior to loading the spooled adhesive bandages into bandage dispenser 100. This preparation step may comprise disposing individual bagged adhesive bandages in an end-to-end configuration between two continuous feeds of material having a single adhesive side, for example a suitable form of tape, whereby each of the two continuous feeds of adhesive material adheres to opposing flat sides of the individual bagged adhesive bandages, and in a manner such that a desired end to be first opened of each bagged adhesive bandage is similarly aligned in the end-to-end configuration. In embodiments, the alignment may be based upon an alignment of one or more pull tabs of the bag, or an alignment of one or more backings of each adhesive bandage, such that upon being ejected from dispensing port 117 a lip or tag of one of the backings may be caused to be initially or fully separated from the dispensed adhesive bandage by separating projection 118. In embodiments, the continuous feed of adhesive bandages may comprise a section of initial feed which may or may not include adhesive bandages, and may instead include a section of leader material which may or may not comprise an adhesive surface. Once prepared, the continuous feed of adhesive bandages may be loaded onto spool 130b prior to disposing spool 130b into bandage dispenser 100.

Upon loading spool 130b into bandage dispenser 100, the initial feed of the continuous feed of adhesive bandages may be loaded into bandage dispenser 100 so as to be fed linearly along the directed pathway illustrated in FIG. 3A, whereby the continuous feed leaves spool 130b and is fed between rollers 140a and 140b, at which point each of the continuous feeds of adhesive materials follow separate paths as shown to collect on spools 130a and 130c. In this manner, the bag of the bagged adhesive bandages approaching rollers 140a,b may be caused to be torn open resulting from a pulling force generated as each of the continuous feeds of adhesive materials follows separate paths to spools 130a,b. Upon being torn open, an individual adhesive bandage may be caused to be dispensed or ejected through dispensing port 117, free of its bag. As the individual adhesive bandage is dispensed, separating projection 118 may catch, and in turn cause to separate, fully or partially, at least one of the backings of the exposed individual adhesive bandage, thus presenting the individual adhesive bandage with a portion of its adhesive surface exposed in a manner where it may be both removed from bandage dispenser 100 and applied in a single-handed operation.

As illustrated by FIG. 3B, bandaged dispenser 200 may perform a method of preparing a continuous feed of individual bagged adhesive bandages in an end-to-end configuration between two continuous feeds of material having a single adhesive side (performed separately in the embodiment of bandage dispenser 100), in addition to performing a method of dispensing an adhesive bandage which may be removed from bandage dispenser 200 and applied in a single-handed operation.

Bandage dispenser 200 may be loaded with two spools of continuous feeds of adhesive material, for example a suitable form of tape, each of which may be initially disposed on spool 230b and spool 230c, such that an adhesive side of the material is oriented outward and away from a center the respective spool onto which it may be loaded. Spool 230b may be disposed in bandage dispenser 200 such that the adhesive side faces stacked plurality of individual bagged adhesive bandages 254 (downward as illustrated), and the continuous feed may be wound around roller 240a to be collected by spool 230a in the directional path illustrated. Spool 230e may be disposed in bandage dispenser 200 such that the adhesive side faces the continuous feed between spool 230b and 230a (upward as illustrated), and the continuous feed may be wound around roller 240b to be collected by spool 230d in the directional path illustrated.

Bandage dispenser 200 may also be loaded with a plurality of individual adhesive bandages arranged in a stacked configuration and disposed in bandage magazine 250 such that a preferred end of each of the plurality of individual adhesive bandages is aligned against a common internal surface of bandage magazine wall 256. In embodiments, the arrangement may be based upon an alignment of one or more pull tabs of the bag, or an alignment of one or more backings of each adhesive bandage, such that upon being ejected from dispensing port 217 a lip or tag of one of the backings may be caused to be initially or fully separated from the dispensed adhesive bandage by separating projection 218.

When stacked plurality of individual bagged adhesive bandages 254 are loaded into bandage dispenser 200, a top-most side of a top-most individual adhesive bandage of the stacked plurality may be brought into interference contact against the adhesive surface of the continuous feed between spools 230b and 230a. In operation, rotation of spool 230a in an anti-clockwise direction will serve to pull the continuous feed and in-turn “grab” the top-most adhesive bandage of the stacked plurality. Upon being fed away from the stack, the continuous feed may proceed to “grab” the next top-most adhesive bandage of the stacked plurality as a result of biasing element 255 biasing the stacked plurality upward and against spool 230b. Similarly, the continuous feed between spool 230c and 230d may be caused to adhere to a side of each individual adhesive bandage opposite of the side adhered to by the continuous feed between spool 230b and 230a. In this manner, a continuous feed of adhesive bandages may be formed by bandage dispenser 200 and prepared for dispensing through dispensing port 217.

The method by which bandage dispenser 200 dispenses an individual bandage may then proceed similarly to that of bandage dispenser 100, whereby the bag of the bagged adhesive bandages approaching rollers 240a,b may be caused to be torn open resulting from a pulling force generated as each of the continuous feeds of adhesive materials following separate paths to spools 230a,d. Upon being torn open, an individual adhesive bandage may be caused to be dispensed or ejected through dispensing port 217, free of its bag. As the individual adhesive bandage is dispensed, separating projection 218 may catch, and in turn cause to separate, fully or partially, at least one of the backings of the exposed individual adhesive bandage, thus presenting the individual adhesive bandage with a portion of its adhesive surface exposed in a manner where it may be both removed from bandage dispenser 200 and applied in a single-handed operation.

Each of the embodiments and methods just described allows single-handed removal and application of an adhesive bandage, with the adhesive bandage remaining in its sterile bag until dispensed. Additionally, each of the embodiments and methods provides for the collection if remnant bandage bags within the housing of bandage dispenser 100,200, with the size and capacity of bandage dispenser 100,200 and its constituent components correlating to a desired initial capacity of adhesive bandages.

Although the present invention and its advantages have been described in detail, it should be understood that various changes, substitutions and alterations may be made herein without departing from the spirit and scope of the invention as defined by the appended claims.

What is claimed is:

1. A continuous-feed adhesive bandage dispenser, comprising:
a housing having a cover;
a plurality of rotating elements disposed between the housing and the cover, wherein the plurality of rotating elements comprises one or more spools and one or more rollers;
a dispensing port located on a surface of the housing; and
a plurality of bagged adhesive bandages disposed within the housing;
wherein each of the plurality of bagged adhesive bandages is ejected from the housing through the dispensing port as a continuous feed of the plurality of bagged adhesive bandages, and wherein one or more paths of the continuous feed is defined by the one or more spools and the one or more rollers.

2. The continuous-feed adhesive bandage dispenser of claim 1, wherein the plurality of bagged adhesive bandages are disposed within the housing as a spooled plurality of adhesive bandages.

3. The continuous-feed adhesive bandage dispense of claim 1, wherein the plurality of bagged adhesive bandages are disposed within the housing as a stacked plurality of adhesive bandages.

4. A method of dispensing a sterile adhesive bandage from a bandage bag, comprising:
providing at least one sterile adhesive bandage, wherein the sterile adhesive bandage is disposed in a bandage bag having two opposing flat surfaces and wherein the two opposing flat surfaces comprise a first flat surface and a second flat surface;
adhering a first material having a single adhesive surface to the first flat surface of the bandage bag;
adhering a second material having a single adhesive surface to the second flat surface of the bandage bag;
dispensing the sterile adhesive bandage from the bandage bag while simultaneously causing the first material to be pulled in a first direction and the second material to be pulled on a second direction.

5. The method of claim 4, wherein the at least one sterile adhesive bandage comprises a plurality of sterile adhesive bandages, each of which is oriented in a same or similar direction.

6. The method of claim 5, wherein the plurality of sterile adhesive bandages form a continuous feed of sterile adhesive bandages.
